(19) United States
(12) Patent Application Publication
(10) Pub. No.: US 2018/0271943 A1
(43) Pub. Date: Sep. 27, 2018

(54) METHODS FOR TREATING INFLAMMATION WITH TGF-BETA

(71) Applicant: Puretein Bioscience LLC, St. Louis Park, MN (US)

(72) Inventors: Brett Casebolt, Edina, MN (US); Ronald E. Strohbehn, Nevada, IA (US)

(21) Appl. No.: 15/544,940

(22) PCT Filed: Jan. 22, 2016
(86) PCT No.: PCT/US2016/014526

§ 371 Date: Jul. 20, 2017

Related U.S. Application Data
(60) Provisional application No. 62/106,855, filed on Jan. 23, 2015.
(60) Provisional application No. 62/110,816, filed on Feb. 2, 2015.

(51) Int. Cl. A61K 38/18; A61K 9/00; A61K 9/06

(52) U.S. Cl. CPC A61K 38/1841; A61K 9/06; A61K 9/0014

(57) ABSTRACT

Provided wherein are methods for treating inflammation. In one embodiment, the method includes administering an effective amount of a composition to a subject having or at risk of having a condition that includes inflammation. The condition can be arthritis, tendonitis, osteoarthritis, fibrosis, shingles, psoriasis, acne, or dermatitis. Examples of dermatitis include atopic dermatitis, contact dermatitis, and seborrhoeic dermatitis. In one embodiment, the method includes administering an effective amount of a composition to a subject having pain, heat, and/or redness associated with inflammation. In one embodiment, the composition administered can include active TGF-β at a concentration of at least 0.001 nanograms/gram (ng/g). Optionally, the active TGF-β can be at a concentration of no greater than 4000 ng/g.

### CROSS-REFERENCE TO RELATED APPLICATIONS

This application claims the benefit of U.S. Provisional Application Ser. No. 62/106,855, filed Jan. 23, 2015, and 62/110,816, filed Feb. 2, 2015, each of which is incorporated by reference herein.

### SUMMARY

Provided wherein are methods for treating inflammation. In one embodiment, the method includes administering an effective amount of a composition to a subject having or at risk of having a condition that includes inflammation. The condition can be arthritis, tendonitis, osteoarthritis, fibrosis, shingles, psoriasis, acne, or dermatitis. Examples of dermatitis include atopic dermatitis, contact dermatitis, and seborrhoeic dermatitis. The inflammation can be acute inflammation, chronic inflammation, or a combination thereof.

In one embodiment, the method includes administering an effective amount of a composition to a subject having pain, heat, and/or redness associated with inflammation. The inflammation can be associated with arthritis, tendonitis, osteoarthritis, fibrosis, shingles, psoriasis, acne, or dermatitis. Examples of dermatitis include atopic dermatitis, contact dermatitis, and seborrhoeic dermatitis. The inflammation can be acute inflammation, chronic inflammation, or a combination thereof. In one embodiment, the treating causes a reduction of pain, heat, redness, or a combination thereof, in an area having inflammation.

The composition administered can include active TGF-β at a concentration of at least 0.001 nanograms/gram (ng/g). Optionally, the active TGF-β can be at a concentration of no greater than 4000 ng/g. In one embodiment, the composition administered can include TGF-β at a concentration of at least 0.001 nanograms/gram (ng/g). Optionally, the TGF-β can be at a concentration of no greater than 4000 ng/g. The TGF-β can be TGF-β1, TGF-β2, TGF-β3, or a combination thereof.

In one embodiment, the administration is topical. In one embodiment, TGF-β present in the composition is obtained from a milk product, such as procream. In one embodiment, the subject is a human, a canine species, a feline species, or an equine species. In one embodiment, the dosage form includes a cream, an ointment, or a lotion.

### BACKGROUND

Transforming growth factor beta (TGF-β, also referred to herein as TGF-B and TGF-beta) is a protein that controls proliferation, cellular differentiation, and other functions in most cells. It is a type of cytokine that plays a role in immunity and wound healing. TGF-β is secreted and exists in at least three forms referred to as TGF-β1, TGF-β2 and TGF-β3. TGF-β was also the original name for TGF-β1, which was the founding member of this family.

TGF-beta is secreted by many cell types, including macrophages, in a latent form in which it is complexed with two other polypeptides, Latent TGF-beta Binding Protein (LTBP) and Latency Associated Peptide (LAP). Serum proteinases such as plasmin catalyze the release of active TGF-beta from the complex. This often occurs on the surface of macrophages where the latent TGF-beta complex is bound to CD36 via its ligand, thrombospondin-1 (TSP-1). Inflammatory stimuli that activate macrophages enhance the release of active TGF-beta by promoting the activation of plasmin. Macrophages can also endocytose IgG-bound latent TGF -beta complexes that are secreted by plasma cells and then release active TGF-beta into the extracellular fluid.

The peptide structures of the three members of the TGF-β family are highly similar. They are all encoded as protein precursors having an N-terminal signal peptide of 20-30 amino acids required for secretion from a cell, a pro-region (called latency associated peptide or LAP), and a C-terminal region that becomes the mature TGF-β molecule following its release from the pro-region by proteolytic cleavage.

### TGF-β Activation

It has been determined that breast milk includes macromolecules having specialized roles in stimulation of growth and have multifunctional regulatory activities. Further, it has been determined that the activity in milk is due to the presence of TGF-β2-like molecules called “milk growth factor” which promotes wound healing responses and growth (Cox and Burk, 1991, Eur. J. Biochem. 197:353-358).

Although TGF-β is important in regulating crucial cellular activities, only a few TGF-β activating pathways are currently known, and the full mechanism behind the suggested activation pathway is not yet well understood. Some of the known activating pathways are cell or tissue specific, while some are seen in multiple cell types and tissues. Proteases, integrins, pH, and reactive oxygen species are just a few of the currently known factors that can activate TGF-β. It is known that perturbations of these activating factors can lead to unregulated TGF-β signaling levels that may cause several complications including inflammation, autoimmune disorders, fibrosis, cancer, and cataracts.

### TGF-β Latency (Latent TGF-β Complex)

All three TGF-βs are synthesized as precursor molecules containing a propeptide region in addition to the TGF-β homodimer. After it is synthesized, the TGF-β homodimer interacts with a Latency Associated Peptide (LAP), a protein derived from the N-terminal region of the TGF-beta gene product, forming a complex called Small Latent Complex (SLC). This complex remains in the cell until it is bound by another protein called Latent TGF-binding Protein (LTBP), forming a larger complex called Large Latent Complex (LLC). The LLC is typically secreted to the extracellular matrix.

In most cases, before the LLC is secreted, the TGF-β precursor is cleaved from the propeptide but it remains attached by noncovalent bonds. After secretion, it remains in the extracellular matrix as an inactivated complex containing both the LTBP and the LAP which needs to be further processed in order to release active TGF-β. Because different cellular mechanisms require distinct levels of TGF-β signaling, the inactive complex of this cytokine gives opportunity for a proper mediation of TGF-β signaling.

### Integrin-Independent TGF-β Activation

The TGF-β activation process can involve the release of the LLC from the matrix, followed by further proteolysis of the LAP to release TGF-β to its receptors. MMP-9 and MMP-2 are known to cleave latent TGF-β. Another means of activation includes acidic conditions which denature the LAP. Treatment of the medium with extremes of pH (1.5 or 12) result in significant activation of TGF beta as shown by radio-receptor assays, while mild acid treatment (pH 4.5) yields far less of the competition achieved by pH 1.5. Some methods of isolating or preparing protein isolates may also activate TGF-β or may concentrate the activated TGF-beta already present. Further, it is speculated that some materials or conditions in the digestive tracts of some mammals may also activate TGF-β.

Structure modification of the LAP can lead to disturbing the interaction between LAP and TGF-β and thus activating it. Factors that may cause such modification may include hydroxyl radicals from reactive oxygen species (ROS). TGF-β is rapidly activated after in vivo radiation exposure ROS.

Finally, levels of Thrombospondin-1 (TSP-1) (a matricellular glycoprotein found in plasma of healthy patients) are known to increase in response to injury and during development. TSP-1 activates latent TGF-β by forming direct interactions with the latent TGF-β complex and inducing a conformational rearrangement preventing it from binding to the matured TGF-β.

The mechanism for the known biological effects of TGF-β 1, 2, and 3 lies in an activation of the molecule from its latent complex to an activated form. It has been theorized that the TGF-β propeptide remains tightly bound to the cytokine after the bonds between the propeptide and the mature TGF-β are cleaved, which renders the growth factor latent. The TGF-β complex may be covalently linked to the extracellular matrix. This latent complexed TGF-β may be considered a molecular sensor that responds to certain signals by releasing the TGF-β (Annes et al., 2003, J. Cell. Sci., 116:217-224).

### BRIEF DESCRIPTION OF THE FIGURES

FIG. 1. A. Pain levels before and after application. B. Duration of pain relief. C. Time to relief. D. Current treatment method of participants indicating that the composition was more effective.

### DETAILED DESCRIPTION OF ILLUSTRATIVE EMBODIMENTS

Provided herein are compositions and methods for using the compositions. A composition provided herein includes TGF-β, and optionally includes other proteins. The TGF-β may be TGF-β1, TGF-β2, TGF-β3, or a combination thereof. In one embodiment, the composition includes TGF-β2. TGF-β associates with distinct binding proteins. Most TGF-β present in products derived from an animal is bound to Latency Associated Peptide and in some cases Latent TGF-binding protein. Since these binding proteins inhibit the activity of TGF-β, most TGF-β present in animal derived products is inactive due to its being bound to binding proteins. For instance, only 5% to 10% of TGF-β2 in plasma or some milk products is not bound to a binding protein and active.

A composition useful in the methods described herein includes active TGF-β1, TGF-β2, TGF-β3, or a combination thereof. Optionally, a composition useful in the methods described herein also includes inactive TGF-β1, TGF-β2, TGF-β3, or a combination thereof. TGF-β proteins are highly conserved between species, and the amino acid sequences of TGF-β proteins from different species are known and readily available to the skilled person. Whether a protein is TGF-β1, TGF-β2, or TGF-β3 can be easily determined by the skilled person. For instance, polyclonal and monoclonal antibodies that specifically bind to TGF-β1, TGF-β2, or TGF-β3 are commercially available, and react with TGF-β1, TGF-β2, or TGF-β3 from various species including human, equine, canine, bovine, and porcine. These readily available antibodies lack cross-reactivity and/or interference by other closely related proteins and binding proteins. Methods for determining whether a TGF-β protein is active are known in the art and routine (Brown et al., 1990, Growth Factors, 3:35-43).

In one embodiment, a composition includes a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration.

A composition compatible with pharmaceutical administration may be prepared by methods well known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include topical (e.g., epicutaneous, transmucosal) administration. Appropriate dosage forms for topical administration may include a cream, ointment, lotion, gel, foam, and skin patch. Methods for making a pharmaceutically acceptable composition that includes TGF-β are known to the skilled person (Juneau et al., U.S. Pat. No. 7,763,257).

Compositions can include sterile aqueous solutions or dispersions and sterile powders for the extemporaneous preparation of sterile solutions or dispersions. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., the TGF-β, such as TFG-β2) in the required amount in an appropriate solvent with one or a combination of ingredients, as required, followed by filtered sterilization. Generally, dispersions are prepared by incorporating the active compound into a sterile vehicle, which contains a basic dispersion medium and any other appropriate ingredients.

A composition for use in topical administration may be formulated into many types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, or pastes (Williams, Transdermal and Topical Drug Delivery, Pharmaceutical Press, London, 2003). Variations and other vehicles will be apparent to the skilled artisan and are appropriate for use in the methods described herein.

It is also contemplated that an active compound may be encapsulated for delivery to a target area such as skin. Non-limiting examples of encapsulation techniques include the use of liposomes, vesicles, and/or nanoparticles (e.g., biodegradable and non-biodegradable colloidal particles that include polymeric materials in which the ingredient is trapped, encapsulated, and/or absorbed—examples include nanospheres and nanocapsules) that can be used as delivery vehicles to deliver such ingredients to skin.

Systemic administration can be by transmucosal delivery. For transdermal administration, penetrants appropriate to the barrier to be permeated are used in the formulation. Such penetrants are generally known in the art, and include, for example, for transmucosal administration, detergents, bile salts, and fusidic acid derivatives. For transdermal administration, the active compounds are formulated into ointments, salves, gels, or creams as generally known in the art.

Pharmaceutical administration can be one or more times per day to one or more times per week, including once every other day. The skilled artisan will appreciate that certain factors may influence the dosage and timing required to effectively treat a subject, including but not limited to the type of condition, the severity of the condition, previous treatments, and the general health and/or age of the subject.

The amount of active TGF-β, such as TGF-β2, to be administered by a topical route in the methods described herein can be determined by standard pharmaceutical procedures in cell cultures or experimental animals, e.g., for determining the ED₅₀ (the dose therapeutically effective in 50% of the population). The data obtained from cell culture assays and animal studies can be used in formulating a range of dosage for use in an animal. The dosage of such compounds lies preferably within a range of circulating concentrations that include the ED₅₀ with little or no toxicity; however, it is expected that high levels of active TGF-β, such as TGF-β2, will not be detrimental to an animal. The dosage may vary within this range depending upon the dosage form employed and the route of administration utilized. For a compound used in the methods of the invention, the therapeutically effective dose can be estimated initially from cell culture assays and/or experimental animals.

TGF-β useful in the methods described herein is obtainable from various sources. In one embodiment, a source is a natural source, such as a biological material from an animal. Examples of animals include, but are not limited to, vertebrates. Examples of vertebrates include, but are not limited to, mammals, such as a species that is bovine, porcine, cervid, canine, feline, equine, ovine, or a human. Another example of a vertebrate is an avian species.

Examples of biological materials include, but are not limited to, blood and blood-derived products, colostrum and colostrum-derived products; egg and egg-derived products (e.g., egg yolk, egg whites, egg membranes), bodily fluids (e.g., saliva, semen), and tissues (e.g., mucosa tissue, intestinal tissue, embryonic tissue). Examples of blood and blood-derived products include, but are not limited to, whole blood, red blood cells, plasma. Examples of milk and milk products include, but are not limited to, whole milk, skim milk, buttermilk, milk protein concentrate, cheese, sweet dairy whey, whey, casein, curd, caseinate and whey products such as whey cream, and procream (also referred to in the art as reduced lactose concentrated whey and whey protein phospholipid concentrate, the milk product collected from a whey filtration process such as microfiltration in the manufacture of whey protein isolate), Whey Protein Concentrate, Whey Protein Isolate, Whey Phospholipids, Reduced Lactose Concentrated Whey, Reduced Lactose Whey, Deproteinized Whey, Whey Cream, Whey Retentate, Whey Protein Hydrolysate, Whey Permeate, Hydrolyzed Whey Protein Concentrate, Lactose alpha-lactalbumin. Examples of colostrum-derived products include, but are not limited to, liquid colostrum whey, colostrum whey protein concentrate, colostrum whey protein isolate, colostrum whey cream, colostrum whey retentate, colostrum procream, colostrum deproteinized whey, colostrum delactosed permeate, colostrum casein, colostrum lactose, colostrum curd. Examples of egg and egg-derived products include, but are not limited to, egg yolk, egg whites, egg membranes. Examples of bodily fluids include, but are not limited to, saliva, semen. Examples of tissues include, but are not limited to, mucosa tissue, intestinal tissue, embryonic tissue. A biological material may be dried.

In one embodiment, the colostrum is colostrum secreted by a female within the first 6, the first 12, the first 24, or the first 48 hours after birth of offspring. In one embodiment, TGF-β useful in the methods described herein is obtained from blood or a blood-derived product. In one embodiment, TGF-β useful in the methods described herein is obtained from a dairy product. In one embodiment, TGF-β useful in the methods described herein is produced using recombinant techniques, or chemically or enzymatically synthesized. As used herein, TGF-β from a natural source, for instance, blood or a blood-derived product, is not produced using recombinant techniques, or chemically or enzymatically synthesized. Biological material that is useful for producing a composition with active TGF-β is readily available commercially.

A biological material may be enriched for the amount of total TGF-β present. A protein is enriched if it is present in a significantly higher fraction compared to the biological material from which the protein was enriched. The higher fraction may be, for instance, an increase of 2-fold, 4-fold, or 6-fold. Enrichment may result from reducing the amount of other molecules present in the biological material, e.g., proteins. However, the term enriched does not imply that there are no other molecules, e.g., proteins, present. Enriched simply means the relative amount of TGF-β has been significantly increased. The term “significant” indicates that the level of increase is useful to the person making such an increase. Enrichment of TGF-β is the result of intervention by a person to elevate the proportion of the protein.

Optionally, TGF-β can be purified from a biological material. A protein is considered to be purified if at least 75%, least 85%, or at least 95% of other components present in the biological material are removed. Proteins that are produced through chemical or recombinant means are considered to be purified. Methods for enriching and/or purifying TGF-β are known to the skilled person and are routine. Non-limiting examples of such procedures include fractionation on immunoaffinity or ion-exchange columns; ethanol precipitation; reverse phase HPLC; chromatography on silica or on an ion-exchange resin such as DEAE; chromatofocusing; SDS-PAGE; ammonium sulfate precipitation; gel filtration using, for example, cross-linked gels and/or hollow fiber; and ligand affinity chromatography.

Much of the TGF-β obtained from natural sources is associated with binding protein, for instance, latency-associated peptide and optionally latent TGF-beta binding protein, which causes the TGF-β to be inactive. The amount of active TGF-β in a composition that is obtained from a natural source can be increased, i.e., the amount of total TGF-β in the composition is unchanged but the amount of active TGF-β as a percentage of the total TGF-β is increased. Methods for increasing the amount of TGF-β that is active include those routinely used to activate functional proteins obtained from a biological material. Such methods include, but are not limited to, exposing the biological material to pH adjustment, heat shock, temperature, alcohol extraction, enzyme addition, pressure, ionic changes, or a combination thereof (Brown et al., 1990, Growth Factors, 3:35-43). Without intending to be limited by theory, such methods typically cause the dissociation of binding protein from the TGF-β protein. In one embodiment, the amount of active TGF-β in a composition that is obtained from a natural source can be increased by at least 2-fold, at least 4-fold, at least 5-fold, or at least 10-fold compared to the concentration of active TGF-β in the composition before it is processed to increase the concentration of active TGF-β. The composition subjected to the processing can be, for instance, a biological material from an animal, such as a milk or milk-derived product. Optionally, the biological material may be one that has been enriched for total TGF-β.

A TGF-β is considered to be active if it is not bound to a binding protein, and is considered to be inactive if it is bound to a binding protein. Active TGF-β is often referred to in the art as free, unbound, bioactive, and/or active. Methods for measuring the concentration of active TGF-β are known to the skilled person and are routine. One example of such a method is an ELISA immunoassay to measure TGF-β2 available from R&D Systems (Minneapolis, Minn., catalog number DB250). Such assays typically include a step of activating all TGF-β present, such as by acid activation and neutralization, followed by immunoassay to measure all TGF-β present. The amounts of inactive and active TGF-β2 can be determined by conducting the immunoassay on a sample without first subjecting the sample to conditions that activate the TGF-β present. The difference in amount of TGF-β in a sample subjected to activation and one not subjected to activation can be used to determine the amount of active TGF-β in the composition.

An example of an acid activation/neutralization is the following. The solutions used are 1 N HCl and 1.2 N NaOH/0.5 M HEPES. To prepare 100 mL of the 1 N HCl solution, slowly add 8.33 mL 12 N HCl to 91.67 mL deionized water, mix well. To prepare 100 mls of the 1.2 N NaOH/0.5 M HEPES solution slowly add 12 mL 10 N NaOH to 75 mL deionized water and mix well. Add 11.9 g HEPES, mix well, and bring final volume to 100 mL with deionized water. The solutions may be stored in polypropylene bottles at room temperature for up to one month. The procedure for activating TGF-β in a sample, such as TGF-β2, is as follows: to 125 mL sample add 25 mL 1 N HCL, mix well; incubate 10 minutes at room temperature; add 25 mL 1.2 N NaOH/0.5 M HEPES, mix well; add 800 mL diluent (e.g., Calibrator Diluent RD5I, available from R&D Systems). Mix well and assay within 2 hours.

Also provided herein are methods for using a composition described herein. In one embodiment, the method is for treating a condition in an animal, such as one or more symptoms of a condition in an animal. The method includes administering an effective amount of a composition described herein to a subject having or at risk of having a condition, or exhibiting symptoms and/or clinical signs of a condition. At least one symptom and/or clinical sign of the condition is changed, preferably, reduced. Optionally, the method includes determining whether at least one symptom and/or clinical sign of the condition is changed, preferably, reduced.

Examples of conditions include, but are not limited to, those associated with or caused by inflammation. Examples of such conditions include, for instance, arthritis, tendonitis, osteoarthritis, fibrosis, cancer, shingles, psoriasis, acne, dermatitis, burns, and wounds. Examples of acne include, but are not limited to, inflammatory acne. Examples of dermatitis (also referred to as eczema) include, but are not limited to, atopic dermatitis, contact dermatitis, and seborrhoeic dermatitis.

Treatment of a condition, symptoms, and/or clinical signs associated with a condition can be prophylactic or, alternatively, can be initiated after the development of a condition. As used herein, the term “symptom” refers to subjective evidence of a condition experienced by the subject. As used herein, the term “clinical sign” or, simply, “sign” refers to objective evidence of a condition. Symptoms and/or clinical signs associated with a condition described herein and the evaluations of such symptoms vary depending upon the condition, and are routine and known in the art. For instance, in some conditions caused by or associated with inflammation, the symptoms and/or signs may include pain, heat (e.g., increased temperature), and/or redness that is localized to an area having inflammation, e.g., arthritis, osteoarthritis, tendonitis. The inflammation may be acute or chronic. Thus, methods of using a composition described herein include reducing pain, reducing heat, and/or reducing redness associated with inflammation.

Treatment that is prophylactic, for instance, initiated before a subject manifests symptoms or signs of a condition, is referred to herein as treatment of a subject that is “at risk” of developing a condition. Thus, typically, an animal “at risk” of developing a condition is an animal having one or more risk factors that are associated with increased risk of having a condition. Risk factors may be correlative or causal. Risk factors for the conditions described herein vary depending upon the conditions and are known to the skilled person. Accordingly, administration of a composition can be performed before, during, or after the subject has manifested symptoms and/or signs of a condition. Whether a subject is responding to treatment may be determined by evaluation of symptoms and/or signs associated with the disease.

In one embodiment, the composition is administered topically. Such a composition may be used in a customary manner. In one embodiment, the amount of active TGF-β in a composition administered topically is is at least 0.001 nanograms active TGF-β per gram of composition (ng/g), at least 0.01 ng/g, at least 0.1 ng/g, at least 1 ng/g, or at least 5 ng/g. In one embodiment, the amount of active TGF-β in a composition administered topically is no greater than 4000 ng/g, no greater than 2500 ng/g, no greater than 1000 ng/g, no greater than 500 ng/g, no greater than 100 ng/g, no greater than 50 ng/g, no greater than 20 ng/g, no greater than 10 ng/g, no greater than 5 ng/g, no greater than 1 ng/g, no greater than 0.1 ng/g, no greater than 0.01 ng/g, or no greater than 0.001 ng/g. For instance, in one embodiment a composition that is applied topically may have active TGF-b present at a final concentration that is between at least 0.001 ng/g and no greater than 20 ng/g, or any combination of concentrations selected from the numbers listed above. The active TGF-β administered may be active TGF-β1, active TGF-β2, active TGF-β3, or a combination thereof. In one embodiment, the active TGF-β administered is active TGF-β2. In one embodiment there is no upper limit on the amount of active TGF-β administered.

In one embodiment, a composition administered topically includes total TGF-β at an amount that is at least 0.001 nanograms TGF-β per gram of composition (ng/g), at least 0.01 ng/g, at least 0.1 ng/g, at least 1 ng/g, or at least 5 ng/g. In one embodiment, the amount of total TGF-β in a composition administered topically is no greater than 4000 ng/g, no greater than 2500 ng/g, no greater than 1000 ng/g, no greater than 500 ng/g, no greater than 100 ng/g, no greater than 50 ng/g, no greater than 20 ng/g, no greater than 10 ng/g, no greater than 5 ng/g, no greater than 1 ng/g, no greater than 0.1 ng/g no greater than 0.01 ng/g or no greater than 0.001 ng/g. For instance, in one embodiment a composition that is applied topically may have total TGF-b present at a final concentration that is between at least 0.001 ng/g and no greater than 20 ng/g, or any combination of concentrations selected from the numbers listed above. The total TGF-β administered may be TGF-β1, TGF-β2, TGF-β3, or a combination thereof. In one embodiment, the TGF-β administered is TGF-β2. In one embodiment there is no upper limit on the amount of TGF-β administered.

The administration can be as needed to treat a condition, symptom, and/or clinical sign, including one or more times a day, weekly, or monthly.

Examples of animals include, but are not limited to, vertebrates. Examples of vertebrates include, but are not limited to, mammals, such as a species that is equine (such as a domesticated horse), canine (such as a domesticated dog), feline (such as a domesticated cat), bovine (such as a domesticated cow), porcine (such as a domesticated pig), cervid (such as a deer), ovine, or a human. Another example of a vertebrate is an avian species (such as domesticated fowl). The animal may be at an age that is between birth and weaning, between post-weaning and pre-adult, or a mature (adult) animal.

A composition described herein may also be administered to a subject in need thereof in combination with other therapeutic compounds to increase the overall therapeutic effect. Therapeutic compounds useful for the treatment of a condition described herein vary depending upon the condition, and such therapeutic compounds are known and routinely used.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Example 1

### Efficacy in Reducing Pain Associated with Inflammation Related Conditions

### Objective:

To measure the efficacy of a composition that includes active TGF-β2 in reducing pain in patients suffering from inflammation related ailments, such as arthritis, tendonitis and other general muscle and joint pains.

### Background/Methods:

An exploratory study was conducted among 52 participants suffering from mild to moderate chronic pain associated with arthritis, tendonitis and general muscle and joint soreness. Participants applied a moisturizing cream containing active TGF-β2 at a concentration of 8.9 nanograms active TGF-β2 per gram of the composition (ng/g) twice daily for three days. Participants reported their pain levels on a scale of 0 to 10 (1 representing no pain and 10 representing maximum pain). Participants completed a detailed survey and provided qualitative post-trial feedback.

### Trial Statistics:

There were 52 participants, 60% female and 40% male. The median age was 59. The primary source of ailment was arthritis (60%, 31 participants), tendonitis (13%, 7 participants), or other ailments (27%, 14 participants). Forty six percent (46%, 24 participants) used pain relievers daily.

### Trial Results:

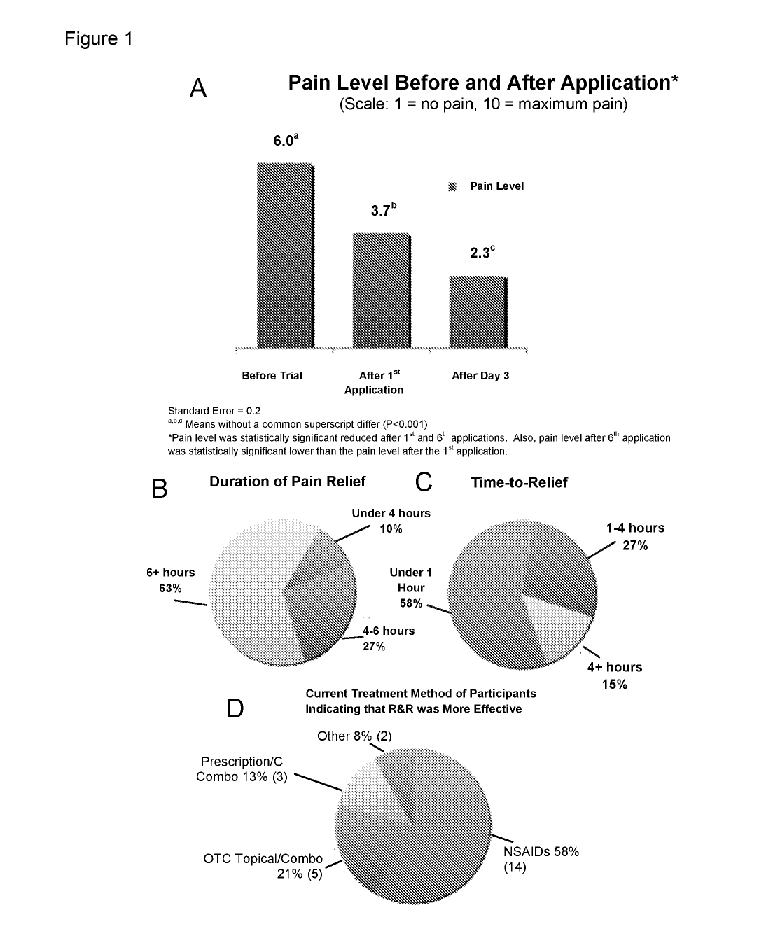

Over the course of the 3 day study: pain levels improved from level 6 to 2 on average; 90% experienced meaningful pain relief; 25% experienced 100% pain reduction; and 68% experienced relief within the first hour (see FIG. 1). The duration of relief was more than 7 hours on average.

Among arthritis participants: 100% (31 participants) experienced a meaningful reduction in pain; there was a 68% pain reduction on average; 73% indicated that the composition was more effective than their current pain relief treatment, which included prescription drugs in several cases.

Among participants using pain relief products daily: 71% reported that the composition was more effective than their current treatment.

Post trial surveys showed positive feedback. The participants indicated superior efficacy or the test composition versus current treatment, positive purchase intent, and willingness to recommend product to others.

### Conclusion:

These data show efficacy of the composition in decreasing pain associated with various inflammation-related conditions, particularly arthritis. Most notably, 90% of trial participants experienced significant pain reduction over the 3 day trial with an average reduction of 67%. Nearly 60% of the trial participants experienced meaningful relief within the first hour of application with relief lasting more than 7 hours on average for the study group. Most participants reported that the test composition was more effective than their existing treatment, including prescriptions and non-steroidal anti-inflammatory drugs (NSAIDs).

### Example 2

### Efficacy in Reducing Pain and Inflammation

### Objective:

To measure the efficacy of a composition in reducing pain and inflammation in patients with arthritis, tendonitis and other chronic inflammation-related ailments. The exploratory study used both thermal imaging data and participant feedback to assess product performance.

### Background:

As described in example 1, a composition that includes active TGF-β2 has been found to significantly reduce pain associated with numerous inflammation-related ailments.

Thermal imaging technology highlights temperature variations near the surface of the skin and is frequently used to identify areas of inflammation associated with arthritis and other chronic pain conditions. Inflammation is characterized by abnormally high temperatures. Thermal imaging analysis can be used to effectively detect these abnormalities and track relative changes over time.

### Methods:

A thermal imaging study was conducted with Vetel Diagnostics (San Luis Obispo, Calif.), an independent thermal imaging specialist. The study involved a before and after thermal imaging analysis of 21 individual cases with inflammation-related pain. Images were taken before and 25 minutes after applying a topical cream containing active TGF-β2 at a concentration of 8.9 nanograms active TGF-β2 per gram of the composition (ng/g). Several controls were used, including the use of a top-selling pain relief cream. Environmental conditions were closely controlled and maintained within an acceptable range.

### Key Thermal Imaging Assumptions:

A temperature differential greater than 1.0 degree between symmetrical left and right sides of body is considered medically significant and highlights potential abnormalities. A delta greater than 0.3 from pre- to post-topical application is considered medically significant. The strongest indicators of performance is when a meaningful change in delta (greater than 0.3) is coupled with participant feedback indicating a significant (>50%) reduction in pain.

### Study Statistics:

There were 21 cases (11 individuals), 7 females and 4 males. The average age was 55, and the primary ailment was osteoarthritis.

### Study Results:

Key findings from topical application of the composition include: reduction in inflammation in more than 70% of cases; 75% reduction in pain on average after 25 minutes; and 90% reduction in pain in nearly 40% of cases.

### Case Studies:

Case 1: Mid 60's female with osteoarthritis on right foot. A red area at the top of the subject's foot indicated a point of pain and inflammation. The thermal temperature changed by 0.8 degrees versus control after treatment (a change greater than 0.3 degrees is considered significant improvement), and the pain level declined from 7 to 3 (1=no pain, 10=maximum pain).

Case 2: Mid 60's male with osteoarthritis in right knee. The outer portion of patient's right knee indicated a point of pain and inflammation. The thermal temperature changed by 0.7 degrees versus control (>0.3 is significant), and the pain level was reduced from 5 to 1 (1=no pain).

Case 3: Mid 40's female with arthritic condition in ball of right foot. The patient's ball of right foot indicated a point of pain and inflammation. The thermal temperature changed by 1.0 degree versus control (>0.3 is significant), and pain level declined from 5 to 1.5 Conclusions:

This study demonstrates a significant reduction in inflammation after topical application of the composition. Participants reported a 75% reduction in pain on average after 25 minutes of application. In the 8 cases when the delta between pre- and post-application was significant (>0.3), the composition reduced reported pain levels by nearly 90% on average.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating a condition comprising inflammation, the method comprising: administering an effective amount of a composition to a subject having or at risk of having a condition comprising inflammation, wherein the composition comprises active TGF-β at a concentration of at least 0.001 nanograms/gram (ng/g) and no greater than 4000 ng/g, wherein the administration is topical.

2. The method of claim 1 wherein TGF-β present in the composition is obtained from a milk product.

3. The method of claim 1 wherein the active TGF-β is TGF-β1, TGF-β2, TGF-β3, or a combination thereof.

4. The method of claim 1 wherein the active TGF-β is active TGF-β2.

5. The method of claim 4 wherein the composition further comprises active TGF-β1, active TGF-β3, or a combination thereof.

6. The method of claim 2 wherein the milk product comprises procream.

7. The method of claim 1 wherein the condition is arthritis, tendonitis, osteoarthritis, fibrosis, shingles, psoriasis, acne, or dermatitis.

8. The method of claim 7 wherein the dermatitis comprises a condition selected from atopic dermatitis, contact dermatitis, and seborrhoeic dermatitis.

9. The method of claim 1 wherein the dosage form comprises a cream, an ointment, or a lotion.

10. The method of claim 1 wherein the treating causes a reduction of pain, a reduction of heat, a reduction of redness, or a combination thereof, in an area localized to a region having inflammation.

11. The method of claim 1 wherein the subject is a human, a canine species, a feline species, or an equine species.

12. A method for treating pain associated with inflammation, the method comprising: administering an effective amount of a composition to a subject in need thereof, wherein the composition comprises active TGF-β at a concentration of at least 0.001 nanograms/gram (ng/g), wherein the administration is topical, wherein pain associated with inflammation is reduced after the administering.

13. The method of claim 12 wherein TGF-β present in the composition is obtained from a milk product.

14. The method of claim 12 wherein the inflammation is acute inflammation.

15. The method of claim 12 wherein the inflammation is chronic inflammation.

16.-24. (canceled)

25. A method for treating a condition comprising inflammation, the method comprising: administering an effective amount of a composition to a subject having or at risk of having a condition comprising inflammation, wherein the composition comprises TGF-β at a concentration of at least 0.001 nanograms/gram (ng/g) and no greater than 4000 ng/g, wherein the administration is topical.

26. The method of claim 25 wherein TGF-β present in the composition is obtained from a milk product.

27.-33. (canceled)

34. A method for treating pain associated with inflammation, the method comprising: administering an effective amount of a composition to a subject in need thereof, wherein the composition comprises TGF-β at a concentration of at least 0.001 nanograms/gram (ng/g), wherein the administration is topical, wherein the pain associated with inflammation is reduced after the administering.

35. The method of claim 34 wherein TGF-β present in the composition is obtained from a milk product.

36. (canceled)

37. The method of claim 34 wherein the inflammation is acute inflammation.

38. The method of claim 34 wherein the inflammation is chronic inflammation.

39.-44. (canceled)
